SEQUENCE LISTING (35 sequences)

SEQ ID NO: 1 - DNA, 5226 bp, Homo sapiens
atgacgtcaagttcgcccgctggcttggaaggttcagacctatcttccatcaacaccatgatgtcggcggtcatgagtgtagggaaggtcacagagaatggcgggagcccccaggggatcaagtccccctcgaagcctccaggaccaaatcggattggcagaaggaaccaggaaacgaaagaggagaagtcttcctataactgccccctgtgtgagaagatttgcactacccagcaccagctgaccatgcacattcgccagcacaacacagacactggaggagccgaccactcatgcagcatctgcggaaagtcactgagctcggccagctccctcgatcgccacatgctggtgcactctggcgagaggccttacaagtgcactgtgtgtggccagtcatttaccaccaatgggaacatgcacagacatatgaagatccatgagaaggaccctaacagtgccacagccacagcccctccatctcctctgaaacgtaggcgattgtcctccaagaggaaactgagtcacgatgccgagtcagagagagaagacccagcaccagctaaaaagatggtagaagacgggcagtcaggtgacttggagaagaaagctgatgaagtctttcactgcccagtatgtttcaaggagtttgtttgcaagtatggactggagacccacatggagacccattcagataacccactaagatgtgacatttgttgtgtcacctttcgaacacatcgaggactgctgcgtcacaacgcgcttgtccacaaacaacttcccagggatgcaatgggcagacctttcatacagaacaacccttcaattcctgctggcttccacgacttaggattcacggacttctcctgtaggaagtttcctcgcatttctcaggcctggtgcgaaacaaacctgcggaggtgcatcagcgagcaacaccgttttgtctgcgacacctgtgacaaggcgttccccatgctctgctcactggctctgcacaagcagacccatgtggcggcagaccagggtcaagaaaagccgcaggccacgcccctgcctggtgacgccctggaccagaagggcttcctggccttgcttggcctgcagcacaccaaagacgtcaggcctgcccccgccgaggagcccctgccggatgacaaccaggcaattcagctccagacactcaagtgtcagctacctcaggaccccggctgcaccaacctgctgagcctgtcacctttcgaagctgcttccctaggcggttctctcacagttctccccgcgaccaaggacagcataaagcacctgtccctgcagcccttccagaagggcttcatcatccagcctgacagcagcattgtggtcaagcccatctctggcgagtcggccatcgagctggcagacatccagcaaattctgaagatggcagcctcggctccccctcagatcagtcttccgcccttctccaaggcccctgccgccccactgcaggcgatcttcaagcacatgccccctctgaagccaaagcccctggtcacaccacggacggtggtggccacctccacgcccccgcctctcatcaacgcccagcaggcttccccgggctgtatcagccccagcctgccgccaccgcccctgaagctcctcaaaggctcagtggaggcggcctccaacgcccacctgctgcagtccaagtccgggacccagccccacgcggccacgcggctctccctgcagcagccgcgggcggagctgccgggccagcctgagatgaagacgcagctggagcaggacagcatcatcgaggccctgctgccgctgagcatggaggccaagatcaagcaggagatcacagagggggaactcaaggccttcatgacagcgcccggcggcaagaagacgcccgccatgcgcaaggtgctctacccctgccgcttctgcaaccaggtgtttgccttctcgggggtcttgcgtgcccacgtgcgctcccacctgggcatctcgccataccagtgcaacatctgcgactacatcgccgccgacaaggccgcgctcatccgccacctgcgcacgcacagtggggagcggccctacatttgcaagatctgccactaccccttcactgtcaaagccaactgcgagcggcacctgcgcaagaagcacctcaaggccacccgcaaggatatcgagaagaacatcgagtatgtgagtagcagcgcggccgagctggtggacgccttctgcgccccggacaccgtgtgccggctgtgcggcgaggacctcaagcactatcgtgccctgcgcatccacatgcgcacgcactgcggccgcggcctgggcgggggccacaagggccgcaagcccttcgagtgcaaggagtgcagcgccgcgttcgcggccaagcgcaactgcatccaccacatcctcaagcagcacctgcacgtgcccgagcaggacatcgagagctacgtgctggccgccgacggcctgggccccgcagaggcgccggccgctgaggcgtcggggcgcggggaggacagtggctgcgctgcccttggtgactgcaagcccctcactgccttcctggaaccccagaacggctttcttcacaggggccccacccagcctccacctccccatgtctcgatcaagttggagcccgccagtagctttgcggtggacttcaatgagcccctggacttctcgcagaagggcctggccctggtccaagtgaagcaggaaaacatctcctttctgagcccttcttccctggtcccctatgactgctccatggagcccatcgacctgtccatccccaagaacttcaggaaaggggacaaggatttggccactcccagcgaagccaagaagcctgaggaggaggcggggagcagcgagcagccctctccctgcccagcacccggcccttctcttcctgtaactttggggcccagcggaatcctggaaagccccatggcccctgctccggcggccaccccggaacccccagcacagcccctgcagggccctgttcagctggcggtcccaatctactcctcagccctggtcagcagccctccactcgtgggcagctcagccctcctgagtggcacagccttgctgcgtccactgcggcccaagcccccgctgcttttgccaaagccccccgtgacagaagagctgcccccgctggcctccattgcccagatcatctcatctgtatcctcggcccccaccctgctgaaaaccaaggtggcggacccagggcccgcaagcactggcagtaacaccacggcttcagacagcttaggaggttctgtccccaaagccgccaccaccgccacccccgctgccaccaccagcccaaaagagtctagtgagcctcccgctccagccagcagcccagaggctgcctctcccaccgagcagggcccagcgggcacgtcgaagaagaggggccggaaaagggggatgaggagccgaccccgcgccaacagcggcggggtggacctggactccagcggggagtttgccagcatcgagaagatgctggccaccacagacaccaacaagttcagtccgtttctgcagacagcggaggacaacactcaggatgaggtggccggagcccctgccgaccaccatgggcccagtgatgaagagcagggcagtcccccagaagacaagctgctgagggccaagcggaactcgtacaccaactgcctgcagaagatcacctgtccccactgtccccgggttttcccttgggccagctccctacagaggcacatgctcacacacactggtcagaaacccttcccttgtcaaaaatgcgatgccttcttttctaccaaatctaactgtgaacgccaccagttgcgcaaacacggagttaccacctgttccctgagaagaaacgggcttatcccccagtcaaaagagagtgatgttggatcccatgatagcacagacagtcagtcggatgcggagactgcagccgccgcgggcgaagtgctagacctcacctcacgggacagagagcagccgtcggagggcgccactgagctccgccaggtcgcaggggatgcgcctgtggagcaggccacggcggaaacggcctcgccggtgcaccgggaagagcacgggcgtggggagagccatgagccggaggaggagcatggcactgaggagagcactggggacgccgacggcgcggaagaggacgcgtcgagcaaccagagcctggacctggacttcgccaccaagctcatggacttcaagctggcggagggcgacggcgaggcaggcgccgggggcgcggcctcgcaggagcagaagctcgcctgcgacacctgtgggaagagcttcaagttcctgggcaccctgagccgccaccggaaggcgcacggccgccaggagcccaaggacgagaagggagatggcgccagcactgcagaggaggggccccagcccgcccctgaacaggaggagaagccccccgagaccccggcagaggtggtggagtcggccccgggtgccggggaggccccggcggaaaagctcgcggaggagacggagggcccctccgacggggagagcgcggccgagaaaaggtcctcagagaagagcgacgatgacaagaaaccaaagacagactcccccaaaagcgtggccagcaaggcagacaagaggaagaaggtctgcagcgtgtgcaacaagcggttctggtcgctgcaggacctgacccggcacatgcgctcccacacaggggaaaggccatacaaatgtcagacctgcgagcgaaccttcaccttgaagcacagcctggttcgccaccagcggatccaccagaaagccaggcatgccaaacaccacgggaaggacagcgacaaggaagagcggggtgaggaggacagcgagaatgagtccacccacagcggcaacaacgccgtctcagagaacgaggctgagctggctcccaatgccagcaaccacatggctgtcacccggagccggaaggagggcttggccagtgccaccaaggactgcagccacagggaggagaaggtcacggcagggtggccgtctgagcctggccagggtgaccttaacccagagagcccggcggccctggggcaggacctgctggagccgcgcagcaagaggcctgcccacccaatcctggccacagctgatggcgcctcccagctcgtggggatggag

SEQ ID NO: 2 - protein, 1742 aa, Homo sapiens
MTSSSPAGLEGSDLSSINTMMSAVMSVGKVTENGGSPQGIKSPSKPPGPNRIGRRNQETKEEKSSYNCPLCEKICTTQHQLTMHIRQHNTDTGGADHSCSICGKSLSSASSLDRHMLVHSGERPYKCTVCGQSFTTNGNMHRHMKIHEKDPNSATATAPPSPLKRRRLSSKRKLSHDAESEREDPAPAKKMVEDGQSGDLEKKADEVFHCPVCFKEFVCKYGLETHMETHSDNPLRCDICCVTFRTHRGLLRHNALVHKQLPRDAMGRPFIQNNPSIPAGFHDLGFTDFSCRKFPRISQAWCETNLRRCISEQHRFVCDTCDKAFPMLCSLALHKQTHVAADQGQEKPQATPLPGDALDQKGFLALLGLQHTKDVRPAPAEEPLPDDNQAIQLQTLKCQLPQDPGCTNLLSLSPFEAASLGGSLTVLPATKDSIKHLSLQPFQKGFIIQPDSSIVVKPISGESAIELADIQQILKMAASAPPQISLPPFSKAPAAPLQAIFKHMPPLKPKPLVTPRTVVATSTPPPLINAQQASPGCISPSLPPPPLKLLKGSVEAASNAHLLQSKSGTQPHAATRLSLQQPRAELPGQPEMKTQLEQDSIIEALLPLSMEAKIKQEITEGELKAFMTAPGGKKTPAMRKVLYPCRFCNQVFAFSGVLRAHVRSHLGISPYQCNICDYIAADKAALIRHLRTHSGERPYICKICHYPFTVKANCERHLRKKHLKATRKDIEKNIEYVSSSAAELVDAFCAPDTVCRLCGEDLKHYRALRIHMRTHCGRGLGGGHKGRKPFECKECSAAFAAKRNCIHHILKQHLHVPEQDIESYVLAADGLGPAEAPAAEASGRGEDSGCAALGDCKPLTAFLEPQNGFLHRGPTQPPPPHVSIKLEPASSFAVDFNEPLDFSQKGLALVQVKQENISFLSPSSLVPYDCSMEPIDLSIPKNFRKGDKDLATPSEAKKPEEEAGSSEQPSPCPAPGPSLPVTLGPSGILESPMAPAPAATPEPPAQPLQGPVQLAVPIYSSALVSSPPLVGSSALLSGTALLRPLRPKPPLLLPKPPVTEELPPLASIAQIISSVSSAPTLLKTKVADPGPASTGSNTTASDSLGGSVPKAATTATPAATTSPKESSEPPAPASSPEAASPTEQGPAGTSKKRGRKRGMRSRPRANSGGVDLDSSGEFASIEKMLATTDTNKFSPFLQTAEDNTQDEVAGAPADHHGPSDEEQGSPPEDKLLRAKRNSYTNCLQKITCPHCPRVFPWASSLQRHMLTHTGQKPFPCQKCDAFFSTKSNCERHQLRKHGVTTCSLRRNGLIPQSKESDVGSHDSTDSQSDAETAAAAGEVLDLTSRDREQPSEGATELRQVAGDAPVEQATAETASPVHREEHGRGESHEPEEEHGTEESTGDADGAEEDASSNQSLDLDFATKLMDFKLAEGDGEAGAGGAASQEQKLACDTCGKSFKFLGTLSRHRKAHGRQEPKDEKGDGASTAEEGPQPAPEQEEKPPETPAEVVESAPGAGEAPAEKLAEETEGPSDGESAAEKRSSEKSDDDKKPKTDSPKSVASKADKRKKVCSVCNKRFWSLQDLTRHMRSHTGERPYKCQTCERTFTLKHSLVRHQRIHQKARHAKHHGKDSDKEERGEEDSENESTHSGNNAVSENEAELAPNASNHMAVTRSRKEGLASATKDCSHREEKVTAGWPSEPGQGDLNPESPAALGQDLLEPRSKRPAHPILATADGASQLVGME

SEQ ID NO: 3 - DNA, 21 bp, Artificial Sequence
  Forward primer-464
tgcttgtcaggggacagatcc

SEQ ID NO: 4 - DNA, 20 bp, Artificial Sequence
  Reverse Primer-464
attggtcaggtgaggggagg

SEQ ID NO: 5 - DNA, 20 bp, Artificial Sequence
  5′hGH
aggaaggcatccaaacgctg

SEQ ID NO: 6 - DNA, 20 bp, Artificial Sequence
  3′hGH
attaggacaaggctggtggg

SEQ ID NO: 7 - DNA, 21 bp, Artificial Sequence
  5′mG3PDH
ggtcatccatgacaactttgg

SEQ ID NO: 8 - DNA, 21 bp, Artificial Sequence
  3′mG3PDH
tcttactccttggaggccatg

SEQ ID NO: 9 - RNA, 21 nt, Artificial Sequence
  siRNA 1
gggcagaccuuucauacaguu

SEQ ID NO: 10 - RNA, 21 nt, Artificial Sequence
  siRNA 2
gaagaaagcugaugaagucuu

SEQ ID NO: 11 - RNA, 21 nt, Artificial Sequence
  targeting firefly luciferase mRNA
cuuacgcugaguacuucgauu

SEQ ID NO: 12 - RNA, 57 nt, Artificial Sequence
  shRNA1
ccggccaggaaacgaaagaggagaacucgaguucuccucuuucguuuccugguuuuu

SEQ ID NO: 13 - RNA, 57 nt, Artificial Sequence
  shRNA2
ccggcgacgaugacaagaaaccaaacucgaguuugguuucuugucaucgucguuuuu

SEQ ID NO: 14 - DNA, 20 bp, Artificial Sequence
  5′hGH
tagaggaaggcatccaaacg

SEQ ID NO: 15 - DNA, 20 bp, Artificial Sequence
  3′hGH
gtctgcttgaagatctgccc

SEQ ID NO: 16 - DNA, 23 bp, Artificial Sequence
  5′mGPA
gccgaatgacaaagaaaagttca

SEQ ID NO: 17 - DNA, 25 bp, Artificial Sequence
  3′mGPA
tcaatagaactcaaaggcacactgt

SEQ ID NO: 18 - DNA, 20 bp, Artificial Sequence
  5′h -Actin
cctgaaccccaaggccaacc

SEQ ID NO: 19 - DNA, 20 bp, Artificial Sequence
  3′h -Actin
cagggatagcacagcctgga

SEQ ID NO: 20 - DNA, 22 bp, Artificial Sequence
  5′RREB1
cgacttaggattcacggacttc

SEQ ID NO: 21 - DNA, 20 bp, Artificial Sequence
  3′RREB1
cagacaaaacggtgttgctc

SEQ ID NO: 22 - DNA, 20 bp, Artificial Sequence
  5′hGATA1
tggcctactacagggacgct

SEQ ID NO: 23 - DNA, 19 bp, Artificial Sequence
  3′hGATA1
catatggtgagccccctgg

SEQ ID NO: 24 - DNA, 25 bp, Artificial Sequence
  5′hG3PDH
caactttggtatcgtggaaggactc

SEQ ID NO: 25 - DNA, 22 bp, Artificial Sequence
  3′hG3PDH
agggatgatgttctggagagcc

SEQ ID NO: 26 - DNA, 19 bp, Homo sapiens
gcccccagcccagccccgt

SEQ ID NO: 27 - DNA, 55 bp, Homo sapiens
gccacaggagaggaacaggagtgatagcccccaaaccccagtcccaccaggccct

SEQ ID NO: 28 - DNA, 29 bp, Homo sapiens
aacaggagtgatagcccccaaaccccagt

SEQ ID NO: 29 - DNA, 29 bp, Artificial Sequence
  ZF2 (mT)
aacaggagtgacagcccccaaaccccagt

SEQ ID NO: 30 - DNA, 29 bp, Artificial Sequence
  ZF2 (3nt)
aacaggagtgacagccggcaaaccccagt

SEQ ID NO: 31 - DNA, 29 bp, Artificial Sequence
  ZF2 (mCC) mutant
aacaggagtgatagccggcaaaccccagt

SEQ ID NO: 32 - DNA, 33 bp, Artificial Sequence
  RREB1 bining sequence on non-globin gene
gatccggtcccccaccatcccccgccatttcca

SEQ ID NO: 33 - DNA, 17 bp, Artificial Sequence
  X1
ttttctccccactttta

SEQ ID NO: 34 - DNA, 20 bp, Artificial Sequence
  X2
cgattccccctcaccagccg

SEQ ID NO: 35 - DNA, 26 bp, Artificial Sequence
  GATA-1 binding sequence
gggacatgataagggagccagcagac

(19) United States
(12) Patent Application Publication
(10) Pub. No.: US 2011/0177033 A1
(43) Pub. Date: Jul. 21, 2011

(54) RAS RESPONSIVE ELEMENT BINDING PROTEIN 1 (RREB1) AS A THERAPEUTIC TARGET FOR THALASSEMIAS AND SICKLE CELL ANEMIA

(75) Inventors: Che-Kun James SHEN, Taipei City (TW); Ruei-Lin Chen, Taipei (TW); Yu-Chi Chou, Taipei (TW)

(73) Assignee: Academia Sinica, Taipei City (TW)

(21) Appl. No.: 13/008,279

(22) Filed: Jan. 18, 2011

Related U.S. Application Data
(60) Provisional application No. 61/296,858, filed on Jan. 20, 2010.

(51) Int. Cl. A61K 35/76; C12Q 1/68; C12N 5/078; A61K 31/7088; A61P 7/06; A61P 7/00

(52) U.S. Cl. 424/93.2; 435/6.19; 435/375; 514/44 A

(57) ABSTRACT

A method of screening compounds capable of activating ζ and/or ε globin gene promoter activity in an erythroid cell is disclosed. The methods comprises contacting in a medium a compound to be screened with RREB1; wherein the medium comprises a polynucleotide comprising the nucleotide sequence of 5′-M-C-M-C-A-M-M-H-M-M-M-3′, wherein M is the nucleotide adenine or cytosine, and H is the nucleotide adenine, cytosine or thymine; the RREB1 bindable to the polynucleotide; determining binding of the compound to the RREB1; and determining change in binding of the RREB1 to the polynucleotide; wherein detection of binding of the compound to the RREB1 and change in binding of the RREB1 to the polynucleotide is indicative that the compound is capable of activating ζ and/or ε globin gene promoter activity. Also disclosed is a method of activating ζ and/or ε globin gene promoter activity in an erythroid cell.

### REFERENCES TO RELATED APPLICATION

The present application claims priority to U.S. Provisional Application Ser. No. 61/296,858, filed Jan. 20, 2010, which is herein incorporated by reference in its entirety.

### FIELD OF THE INVENTION

The present invention relates generally to anemia, and more specifically to anemia disease genes targeting.

### BACKGROUND OF THE INVENTION

The human α-like (5′-ζ(embryonic)-α2-α1 (fetal/adult)-θ1 (fetal/adult)-3′) and β-like (5′-ε (embryonic)-^{G}γ(fetal)-^{A}γ(fetal)-δ-β(adult)-3′) globin gene clusters each extend over 50 kb on chromosomes 16 and 11, respectively. Expressions of the genes within both clusters in erythroid cells are under temporal control during development, with reciprocal silencing of the embryonic/fetal globin genes and induction of the fetal/adult globin genes in a gene-order manner (hemoglobin switch). The coordinated hemoglobin switch processes of the two clusters are also accompanied with shifting of the hematopoiesis sites. A number of previous studies have shown that the erythroid tissue- and developmental stage-specific expressions of the mammalian globin gene clusters including those of the humans are regulated by a variety of different protein-DNA and protein-protein complexes formed at different DNA sequence motifs within the globin gene promoters and their upstream regulatory elements (URE), i.e., the β-LCR and α-HS-40. These proteins include transcription factors serving as either activators or repressors, which include GATA1, NF-E2, EKLF, YY1, TR2/TR4, NF-E4 and BCL11A, etc.

Identification and detailed analysis of the transcription repressors of the embryonic/fetal globin genes would allow the design of appropriate therapeutic approached to re-turn on these genes, thus substitute for the functioning of the defective/silenced/deleted adult α or β globin gene in sickle cell anemia and severe thalassemia.

### SUMMARY OF THE INVENTION

In one aspect, the invention relates to a method of screening compounds capable of activating ζ and/or ε globin gene promoter activity in an erythroid cell. The method comprises: a) contacting in a medium a compound to be screened with RREB1; wherein the medium comprises a polynucleotide comprising the nucleotide sequence of 5′-M-C-M-C-A-M-M-H-M-M-M-3′, wherein M is the nucleotide adenine or cytosine, and H is the nucleotide adenine, cytosine or thymine; the RREB1 bindable to the polynucleotide; b) determining binding of the compound to the RREB1; and c) determining change in binding of the RREB1 to the polynucleotide; wherein detection of binding of the compound to the RREB1 and change in binding of the RREB1 to the polynucleotide is indicative that the compound is capable of activating the ζ and/or ε globin gene promoter activity in the erythroid cell.

In another aspect, the invention relates to a method of activating ζ and/or ε globin gene promoter activity in an erythroid cell. The method comprises contacting the cell with a composition comprising a nucleic acid corresponding to the sequence of SEQ ID NO: 9, SEQ ID NO: 10, SEQ ID NO: 12, or SEQ ID NO: 13, thereby activating the ζ and/or ε globin gene promoter activity in the erythroid cell.

Further in another aspect, the invention relates to a method of treating a subject with thalassemias and/or sickle cell anemia. The method comprises administering to the subject a vector expressing a nucleic acid corresponding to the sequence of SEQ ID NO: 12 or SEQ ID NO: 13, thereby treating the subject with thalassemias and/or sickle cell anemia.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1A is a schematic illustration of the α-like globin locus and the ζ globin promoter region. The physical maps of the α-like globin gene cluster and the protein binding sites in the ζ globin promoter are shown. Shown below the ζ globin promoter is the ZF2 motif. The slash bar indicates the position of the ZF2 motif as mapped previously by footprinting analysis.

FIG. 1B is a diagram of promoter-reporter construct. The reporter is the human growth hormone (hGH) as driven by the human ζ globin promoter (ζ) cis-linked with the HS-40 enhancer.

FIG. 1C shows the sequences of ZF2 and its mutants. The lower case alphabets represent the mutated nucleotides in ZF2. The consensus GATA1 and RREB1 sequences are boxed. Also listed is the consensus sequence of RREB1 binding sites.

FIGS. 1D-1E show assessment of ζ globin promoter activity in erythroid cell cultures. At 48 hrs or 96 hrs after transfection, the culture media were collected and the hGH levels were determined by radioimmunoassay. Star marks the p value (<0.05). The data were derived from three independent experiments.

FIGS. 2A-2E show analysis of transgenic mice. FIG. 2A-2B. Generation of the transgenic mice. FIG. 2A is a diagram of the Xho I-Not I DNA fragment used for generation of the transgenic mice. The probes (PA1 and PA3) used for genotyping by Southern blotting are indicated by underlines under the reporter map. The copy numbers of the transgene were determined by Southern blotting as exemplified in FIG. 2B. The genomic DNAs from the tails were digested with BamH I and then hybridized with the probes. The DNA sizes of the markers are indicated on the right sides of the blots. The position of the head-to-tail tandem repeats of the transgene are marked on the side of the left blot. The endogenous DNA methyltransferase gene (MT) serves as the loading control on the blots. For copy number determination of the transgene, known copies of BamH I-digested Xho I-Not I fragment were loaded on gel and probed with PA3 (right panel). FIG. 2C. Tissue-specific expression patterns of the wt and mCC (mt) ζ globin promoters in transgenic mice. The phenylhydrazine treated, anemic mice were sacrificed and the total RNAs were purified from several different adult tissues. The levels of the hGH RNAs were determined by semi-quantitative RT-PCR using mouse G3PDH as the internal control. B, blood; S, spleen; L, liver; K, kidney; Br, brain. FIG. 2D-2E. Quantitative RT-PCR analysis of hGH mRNA in total RNAs isolated from E9.5 embryos and E14.5 fetal livers. Note the higher hGH mRNA levels in samples with mutant human ζ globin promoter transgenes.

FIGS. 3A-3H show EMSA of factor-binding on the ZF2 motif. The identification of factors binding to the ZF2 motif in nuclear extract was analyzed by EMSA. FIG. 3A. Nucleotide sequences of the oligos used for EMSA. Only one strand of each oligo is shown. The GATA1 and RREB1-binding sequences on the wild type ZF2 are indicated. FIG. 3B. Formation of DNA-protein complexes in nuclear extracts prepared from K562 (K), uninduced MEL (UM), HeLa (H), and 293T (T) cells. The four slow-migrating DNA-protein complexes formed in the K562 (K) extracts, a, b, G, and ? are indicated. Note the presence of complex “G” only in the “K” and “UM” lanes. Also the complex “a” is absent when the ZF2 (mCC) or ZF2 (3 nt) oligo was used as the probe. FIG. 3C. Competition among different ZF2 oligos. The factor-binding specificities on the ZF2 motif in K562 nuclear extract was carried out with or without the presence of 100-fold molar excess of unlabeled oligos, as indicated in the figure as the competitors. For more details, see text. FIG. 3D. Competition between ZF2 and GATA1 oligos in EMSA. Both the K562 (K) and uninduced MEL (UM) extracts were used. Note that complex “G”, but not complex “a”, disappeared ( [symbol] ) in the presence of 100-fold molar excess of cold GATA1 oligo. FIG. 3E. Supershift assay using anti-GATA1. Nuclear extracts from three different cell types were prepared as described above, preincubated with the anti-GATA1 antibody, and then used in EMSA. Note the disappearance ( [symbol] ) of band “G”, but not band “a” or band “b”, upon pre-incubation with anti-GATA1 (lanes 5-7). FIG. 3F. Competition between ZF2 and RREB1 oligos in EMSA. Note that complex “a”, but not complex “G” or “b”, formed on the ZF2 (wt) oligo disappeared upon use of 100-200-fold molar excess of cold RREB1 oligo (lanes 5 and 6). All three complexes disappeared in the presence of cold ZF2 (wt) oligo (lanes 3 and 4). FIG. 3G. Competition among ZF2, RREB1, X1, and X2 oligos. Note that complex “a” formed on the ZF2 oligo was competed out by excess of cold ZF2 (lane 2) or RREB1 oligo (lane 3), but not by 100-fold molar excess of X1 (lane 4) or X2 (lane 5). FIG. 3H. Supershift assay using anti-Myc. Left panel, EMSA patterns using the ZF2 (wt) oligo (lanes 1-3) or ZF2 (mCC) oligo (lane 4) and nuclear extracts prepared from K562 cells transfected with pEF-Myc vector (lane 2) and pEF-Myc-RREB1 (lanes 3 and 4), respectively. Note the increase of the complex band “a” in lane 3. Right panel, patterns of EMSA using the ZF2 oligo and Myc-RREB1 overexpressing K562 nuclear extract without (lane 5) or with preincubation with increasing amounts of the anti-Myc antibody (lanes 6-8).

FIGS. 4A-4C show the expressional levels of α-like globin genes in RREB1-depleted cells. FIG. 4A. siRNA oligos were transiently transfected into K562 cells by electroporation. The cells were collected at 48 hrs later to purify the RNA for analysis. The remnants were re-electroporated with the same siRNA oligos again. The upper histogram represents the data after 48 hrs of transfection. The bottom panel consists of data at 96 hrs post-transfection after the two sequential transfections of the RNAi oligos. A luciferase siRNA was used as the non-specific control. Two independent siRNA oligos targeted to the RREB1 mRNA were used. FIG. 4B. Lentiviral-mediated knock-down of RREB1 mRNA in K562 cells. Cells were infected with the indicated lentiviruses and then collected on the 10^{th} day after viral infection for RNA analysis by quantitative RT-PCR. The panel shows the level of the RREB1 protein, as analyzed by Western blotting (WB). FIG. 4C. Lentiviral-mediated knock-down of RREB1 mRNA in primary human erythroid cells. Primary cultures of human erythroid cells were infected with lentivirus carrying shRNA2 targeting the RREB1 mRNA as described in the Materials and Methods. The total RNAs were isolated at 10^{th} day post-infection and subjected to quantitative RT-PCR analysis. FIG. 4C shows the mRNA levels of α-globin locus genes.

FIG. 5 shows Lentiviral-mediated knock-down of RREB1 mRNA in human erythroid K562 cells.

FIG. 6 shows RNAi knock-down of RREB1 increased the level of embryonic ε globin gene expression in human primary erythroid culture.

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

In one aspect, the invention relates to a method of screening compounds capable of activating ζ and/or ε globin gene promoter activity in an erythroid cell. The method comprises: a) contacting in a medium a compound to be screened with RREB1; wherein the medium comprises a polynucleotide comprising the nucleotide sequence of 5′-M-C-M-C-A-M-M-H-M-M-M-3′, wherein M is the nucleotide adenine or cytosine, and H is the nucleotide adenine, cytosine or thymine; the RREB1 bindable to the polynucleotide; b) determining binding of the compound to the RREB1; and c) determining change in binding of the RREB1 to the polynucleotide; wherein detection of binding of the compound to the RREB1 and change in binding of the RREB1 to the polynucleotide is indicative that the compound is capable of activating the ζ and/or ε globin gene promoter activity in the erythroid cell.

In one embodiment of the invention, the aforementioned method is for identifying a compound capable of increasing ζ and/or ε globin gene promoter activity in an erythroid cell, in which the method comprises detecting binding of the compound to be screened to the RREB1 and inhibition of binding of the RREB1 to the polynucleotide.

In another embodiment of the invention, the compound is capable of increasing expression of two or more than two globin genes chosen from ζ globin gene, ε globin gene and α globin gene.

In another embodiment of the invention, the compound is capable of increasing expression of ζ globin gene, ε globin gene and α globin gene.

In another embodiment of the invention, the compound is capable of increasing expression of ζ globin gene and α globin gene.

In another embodiment of the invention, the compound is capable of increasing expression of ζ globin and ε globin genes.

In another embodiment of the invention, the aforementioned method is for screening antianemic agents.

In another embodiment of the invention, the aforementioned method is for screening agents for treating thalassemias and/or sickle cell anemia.

In another embodiment of the invention, the polynucleotide is replaced by a DNA comprising the nucleotide sequence of SEQ ID NO: 26.

In another embodiment of the invention, the polynucleotide is replaced by a DNA comprising the nucleotide sequence of SEQ ID NO: 28.

In another aspect, the invention relates to a method of activating ζ and/or ε globin gene promoter activity in an erythroid cell. The method comprises contacting the cell with a composition comprising a nucleic acid corresponding to the sequence of SEQ ID NO: 9, SEQ ID NO: 10, SEQ ID NO: 12, or SEQ ID NO: 13, thereby activating the ζ and/or ε globin gene promoter activity in the erythroid cell.

Further in another aspect, the invention relates to a method of treating a subject with thalassemias and/or sickle cell anemia. The method comprises administering to the subject a vector expressing a nucleic acid corresponding to the sequence of SEQ ID NO: 12 or SEQ ID NO: 13, thereby treating the subject with thalassemias and/or sickle cell anemia.

In one embodiment of the invention, the vector is a lentiviral vector.

In another embodiment of the invention, the nucleic acid is a short hairpin RNA (shRNA).

Further in another embodiment of the invention, the nucleic acid is a short interfering RNA (siRNA).

Yet in another embodiment of the invention, the administering decreases the amount of RREB-1 protein expressed by the erythroid cell.

The invention relates to the discovery that the activity of the HS-40-linked ζ globin promoter with the ZF2 mutated is relatively higher than the wild type ζ promoter in transgenic mice. With combined use of transient transfection, site-directed mutagenesis and electrophoretic mobility shift assays, attempts have been made to identify the putative factor(s) binding to the ZF2 motif. These assays together with RNAi knock-down experiments suggest that RREB1 is one of the factors repressing the ζ globin promoter activity through binding to the ZF2 motif.

The invention relates to the transcription repressors of embryonic/fetal globin genes and the design of therapeutic approaches to re-turning on these genes to substitute for the functioning of the defective/silenced/deleted adult α or β globin gene in sickle cell anemia and severe thalassemia. Using a variety of molecular, cellular, and transgenic mice technologies, it was discovered that the protein RREB1 is a repressor responsible for developmental silencing of the human embryonic ζ and ε globin gene expressions. The invention relates to RREB1 for use as the target for designing of new therapeutic approaches and development of new drugs to return-on the expression of human ζ and ε globin gene expressions in fetal/adult erythroid cells of patients with thalassemias or sickle cell anemia.

The mammalian embryonic ζ globin genes, including that of the humans, are expressed at the early embryonic stage and then switched off during erythroid development. This autonomous silencing of the ζ globin gene transcription is likely regulated by the co-operative works among various protein-DNA and protein-protein complexes formed at the ζ globin promoter and its upstream enhancer (HS-40). It was discovered by the inventors that a protein-binding motif, ZF2, contributes to the repression of the HS-40 regulated human ζ promoter activity in erythroid cell lines and in transgenic mice. Combined site-directed mutagenesis and electrophoretic mobility shift assay (EMSA) suggest that repression of the human ζ globin promoter is mediated through binding of the zinc-finger factor RREB1 to ZF2. This model is further supported by the observation that the human ζ globin gene transcription is elevated in human erythroid K562 cell line or the primary erythroid culture upon RNAi knock-down of RREB1 expression. These data together suggest that RREB1 is a putative repressor for the silencing of the mammalian ζ globin genes during erythroid development. Since ζ globin is a powerful inhibitor of HbS polymerization, our experiments have provided a foundation for therapeutic upregulation of ζ globin gene expression in patients with severe hemoglobinopathies.

Compound binding of RREB1. Compound binding properties of RREB1 are assessed using purified 6His-v5 tagged compound immobilized to a NICKEL-SEPHAROSE™ column (via the 6His tag). Compounds are passed through the column (e.g., 50 μl of 100 nM) in the presence of 10 mM NH₄OAc (pH 7.4) and 10% MeOH. Flow through is analyzed for compound content by mass spectroscopy. Some compounds may give a stronger signal by MS, signal strengths will be normalized to show the effects of RREB1 on compound retention. In the presence of RREB1, if some selected compounds have a much longer retention time in the column, it will indicate a stronger association of these particular compounds with RREB1. When marked increases in retention times are only seen with a subset of compounds, it indicates that not only does RREB1 bind compounds, but it also displays selectively in the compounds it binds.

A high throughput assay for measuring RREB1 DNA binding. The assay is used for determining the effects of molecules on the association of RREB1 with double stranded DNA (dsDNA). This assay uses fluorescence polarization to measure the interaction of RREB1 with dsDNA. This assay has 4 components:

i) a dsDNA comprising RREB1 binding consensus sequence: 5′-M-C-M-C-A-M-M-H-M-M-M-3′, wherein M is the nucleotide adenine (A) or cytosine (C), and H is the nucleotide adenine (A), cytosine (C) or thymine (T).

Both the sense and antisense strands are labeled with FITC at the 3′ end using a 6 carbon spacer.

ii) Purified RREB1. Human RREB1 tagged at the amino terminus with the 6His and v5 tags is synthesized in baculovirus and purified using NI²⁺-SEPHAROSE™ chromatography.

iii) reaction buffer (1×):100 mN Tris HCl (pH 7.5), 800 mM NaCl, 10 mM EDTA, 100 mM β-mercaptoethanol, 1% (w/v) TWEEN-20™.

iv) Fluorescence polarization plate reader (TECAN POLARION™ or other equivalent device).

Methodology. A mastermix is prepared consisting of 1× reaction buffer, 2 nM labeled oligo and 0.125 or 0.250 μg RREB-1 per 100 μl of mastermix. Compounds are added to the bottom of a 96 well plate. 100 μl of mastermix is added to each well and allowed to equilibrate for 30 sec. fluorescence polarization is then measured (see U.S. Pat. No. 7,851,153, which is herein incorporated by reference in its entirety).

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Methods and Materials

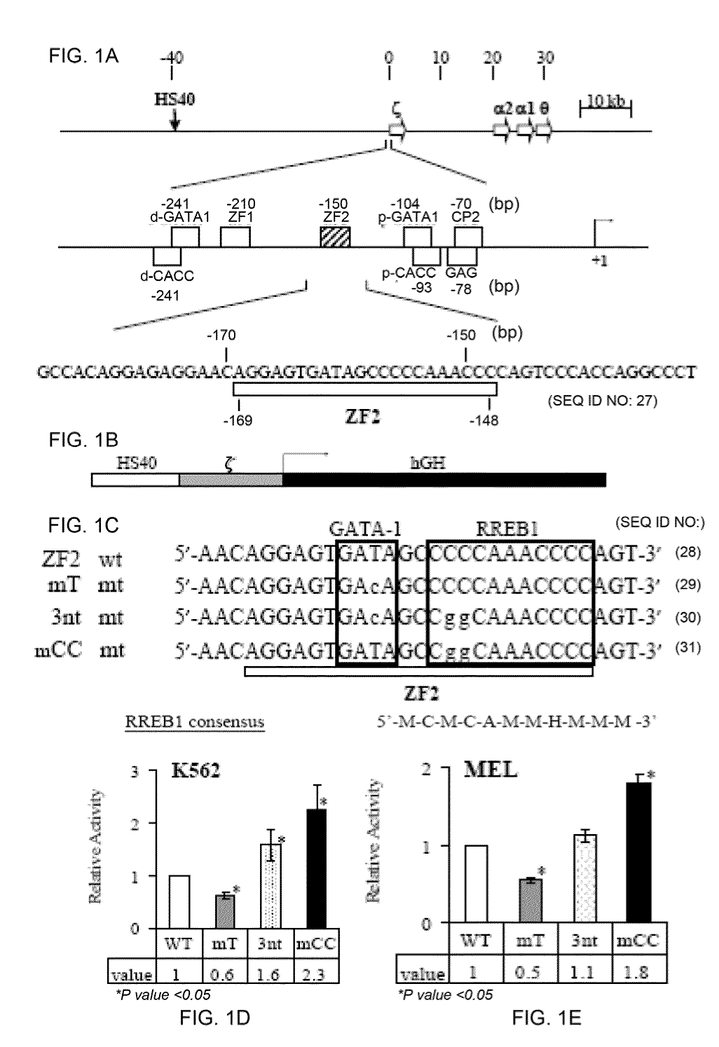

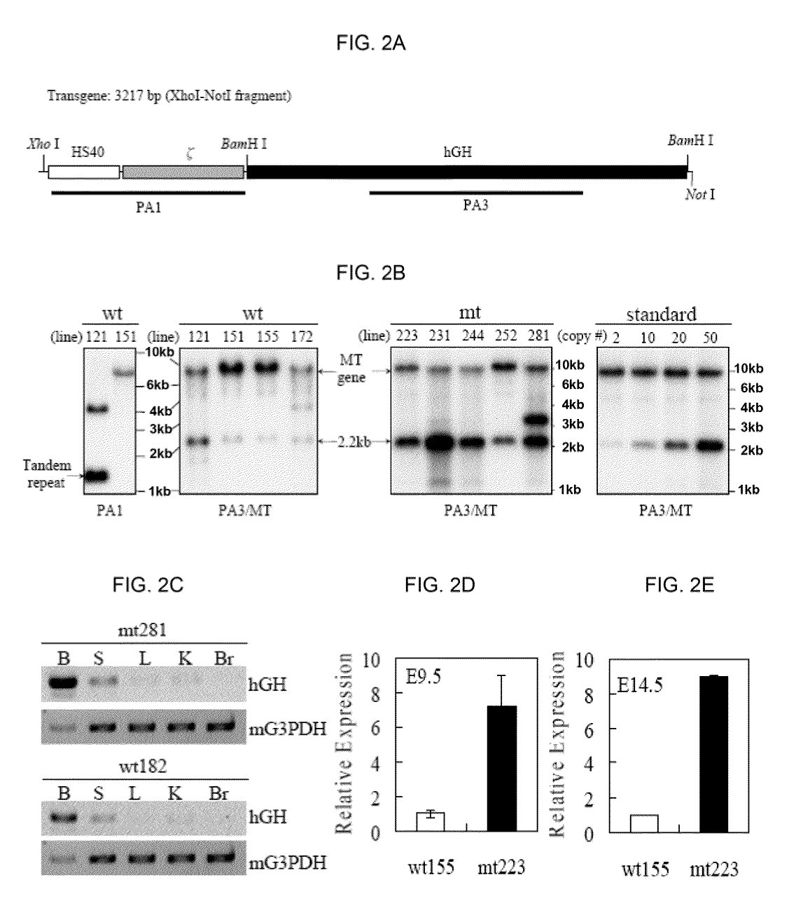

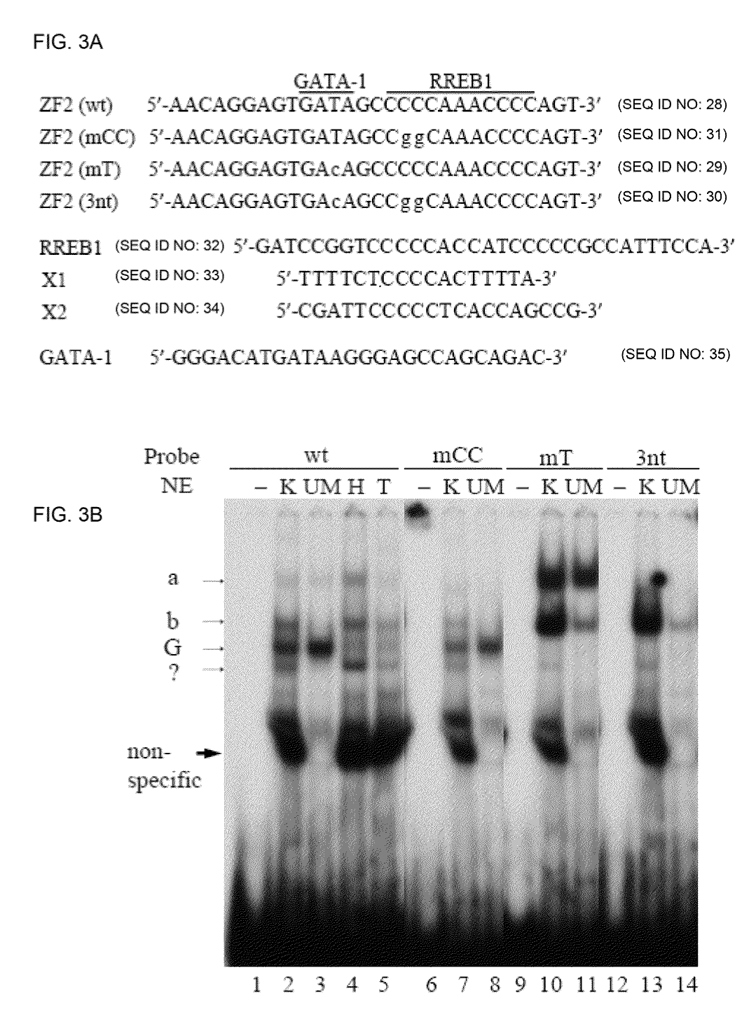

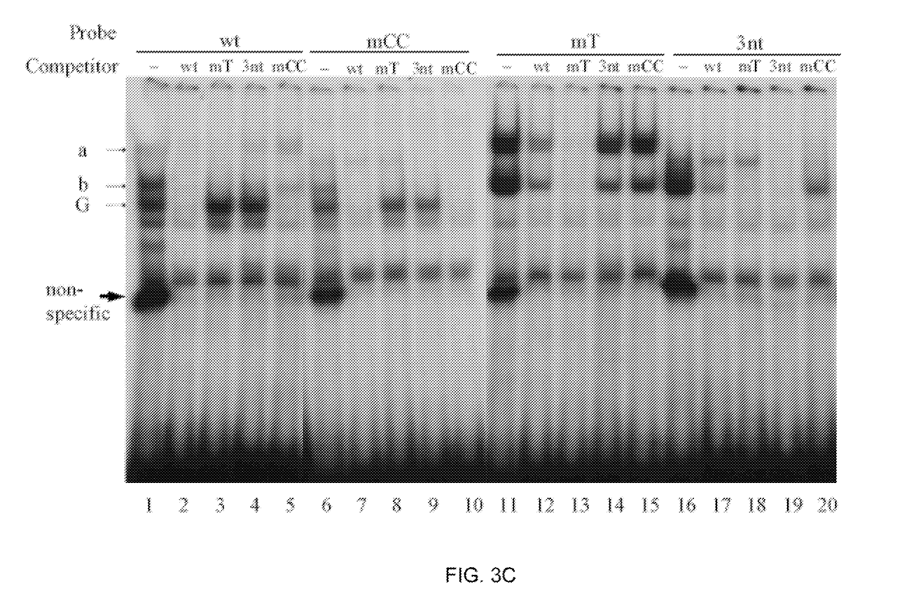

Plasmids. The construct pBS-HS40-ζ-hGH (FIG. 1B) described previously was used in the current study but with replacement of the backbone with that of pBluescript II KS (−) (Stratagene) (21). This new pBS-HS40-ζ-hGH plasmid was then used as the parental plasmid to introduce different mutations into the ζ globin promoter with use of the QUIKCHANGE™ site-directed mutagenesis kit from STRATAGENE™. pBS-HS40-α-hGH was generated by replacement of the ζ globin promoter in pBS-HS40-ζ-hGH with a 1.5 kb Pst I fragment of the α globin promoter. pEF-Myc-RREB1 was constructed by cloning of the RREB1 cDNA (GenBank accession no.: NM_{—}001003699; SEQ ID NO: 1) amplified by PCR using the PFUULTRA™ II Fusion HS DNA Polymerase (STRATAGENE™), in the Sal I/Xho I sites of the pEF/myc/cyto vector (INVITROGEN™). RREB1 protein sequence is listed as SEQ ID NO: 2.

Cell cultures and DNA transfection. K562 cells were maintained in RPMI 1640 medium supplemented with 10% FBS and 1% penicillin-streptomycin in a 37° C. chamber under a 5% CO₂ humidified atmosphere. MEL, HeLa and 293T cells were cultured in the same condition but in DMEM (GIBCO). For DNA transfections, the cells were harvested at the density of 5-8×10⁵ (K562) or 8−10×10⁵ (MEL, 293T and HeLa) per ml. The transfection was carried out using NEON™ transfection system (INVITROGEN™). 2×10⁶ cells were transfected with 5 μg, of the test plasmids and 1 μg of pCMV-β-gal. Following microporation, the K562 cells were seeded in 6-well plates with 5 ml antibiotics-free RPMI for 48 hrs before the hGH assay. The MEL cells were seeded with 5 ml antibiotics-free DMEM for 24 hrs and then induced with 2% DMSO for 96 hrs before the hGH assay. The 293T and HeLa cells were seeded in 6-well plates with 5 ml antibiotics-free DMEM for 48 hrs before the hGH assay.

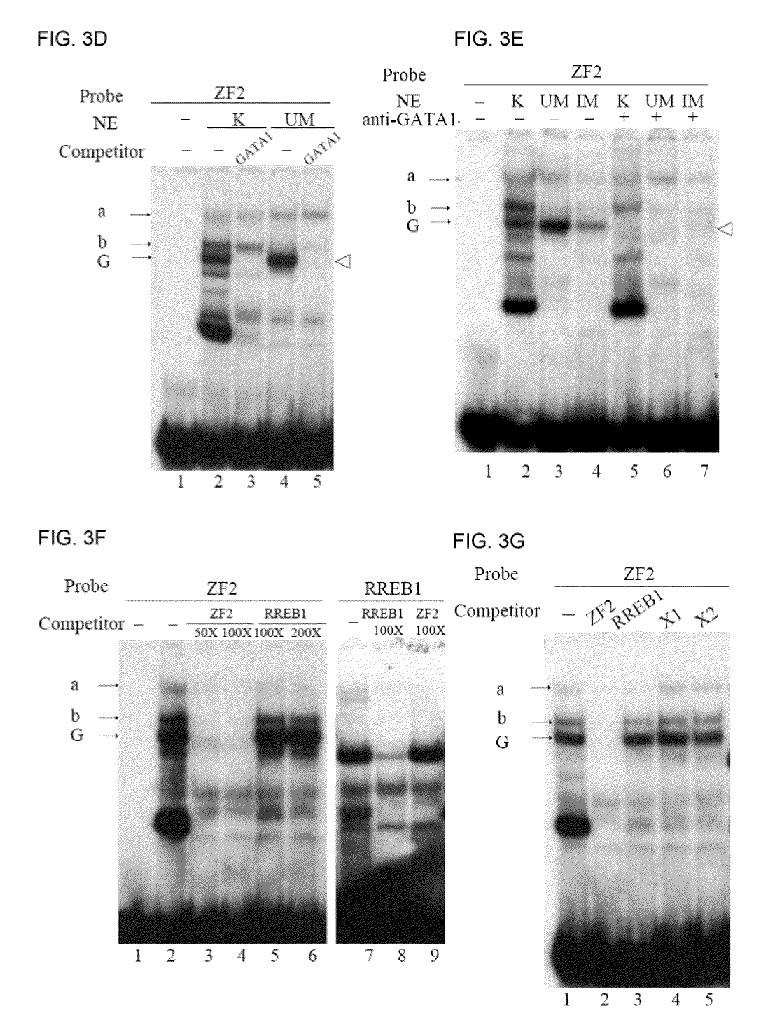

Generation and genotype analysis of transgenic mice. The transgenic mice were generated in the transgenic core facility at IMB using the standard pronuclei microinjection method. The Xho I-Not I DNA fragments isolated from the pBS-HS40-ζ-GH plasmid series (see FIGS. 1B-1C) were used for microinjection. For genotyping of the mouse tail DNAs, the transgene was detected with use of the primers (5′-TGCTTGTCAGGGGACAGATCC-3% SEQ ID NO: 3 and 5′-ATTGGTCAGGTGAGGGGAGG-3% SEQ ID NO: 4) which amplified a 464 bp product. For further structural and copy number analysis, the tail DNAs were digested with BamH I and analyzed by Southern blotting with several probes. The PA1 probe (1,026 bp) hybridized to the 5′-end of the transgene while the PA3 probe (1,051 bp) hybridized to a 2.2 kb BamH I fragment. The 960 bp MT probe from the DNA methyltransferase I gene was used as the loading control. The Xho I-Not I fragment of transgene was also used as the copy-number standard in the Southern blotting analysis (FIG. 2B). After PA1 hybridization, the integrity and the single-copy nature of the transgene could be determined. Use of PA3 probe and comparison of the hybridization intensities to those of the copy-number standard blot provided the copy numbers of the transgene in different lines (Table 2). Quantitation of the band intensities on the blots was carried out in a Fuji FLA-5000 Phosphoimager.

Semi-quantitative RT-PCR analysis. For induction of anemia, 8-month-old mice were injected with phenylhydrazine (40 μg/g of body weight) twice separated by 8 hrs. The treated mice were sacrificed on the sixth day and the tissue RNAs were isolated by Trizol reagent (Invitrogen). Each RT reaction was performed with use of SuperScript II reverse transcriptase (Invitrogen) and 1 μg of RNA. One-tenth of the RT products was used as the template in PCR (Fermentas). The amplifications were carried out at the thermal cycle of 94° C. for 30 sec, 58° C. for 30 sec, and 72° C. for 30 sec. The products of hGH and mouse glyceraldehydes 3-phosphate dehydrogenase (G3PDH) are 313 bp and 525 bp, respectively. The sequences of the primers are as follows: 5′hGH, 5′-AGGAAGGCATCCAAACGCTG-3′ (SEQ ID NO: 5); 3′hGH, 5′-ATTAGGACAAGGCTGGTGGG-3′ (SEQ ID NO: 6); 5′mG3PDH, 5′-GGTCATCCATGACAACTTTGG-3′ (SEQ ID NO: 7); 3′mG3PDH, 5′-TCTTACTCCTTGGAGGCCATG-3′ (SEQ ID NO: 8).

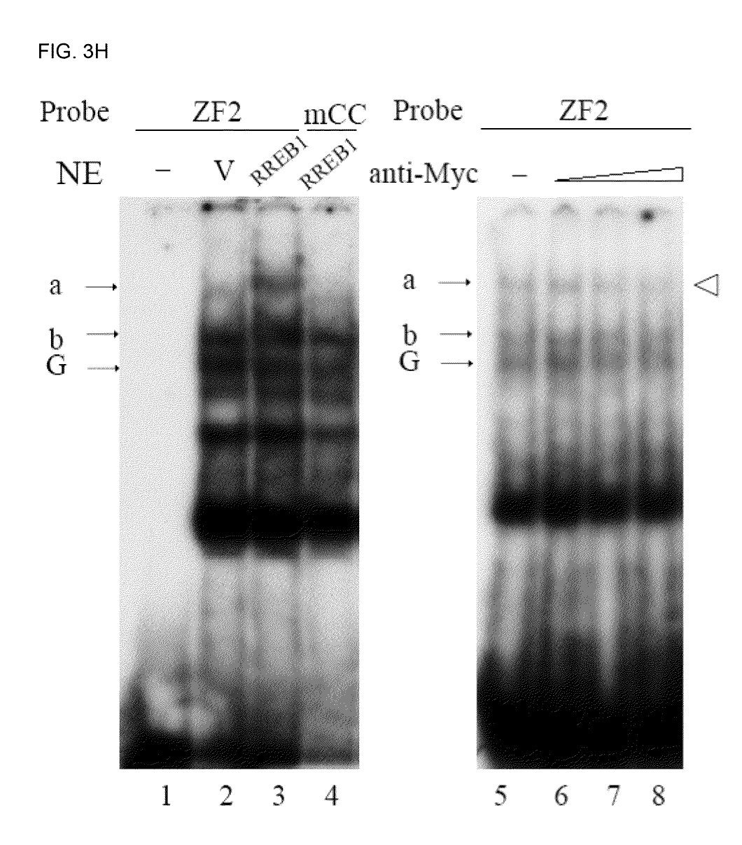

Electrophoretic mobility shift assays (EMSA). Nuclear extracts were prepared from K562, MEL, HeLa and 293T cells as described previously, in the presence of protease inhibitors (Roche, Protease Inhibitor Cocktail Tablets). The oligos used are listed in FIG. 3A. All of the DNA binding reactions were performed as described by Wen et aL with minor modification (Wen, S. C. et al., (2000) Mol Cell Biol 20, 1993-2003). The double-stranded probes were 5′-end labeled with ³²P by T4 polynucleotide kinase (NEB), and then purified over a Sephadex G-25 column (Roche). 5 or 10 μg, of the nuclear extracts were incubated with the probe (100,000 cpm) at room temperature for 15 min in 20 μl of 20 mM HEPES [pH 7.9], 50 mM KCl, 1 mM MgCl₂, 0.5 mM dithiothreitol, 4% glycerol and 1 μg of poly (dI-dC). For competition EMSA, excess of cold oligos were used (see legends of FIGS. 3A-3H for more details).

For further identification of the complexes by supershift assay, the antibody anti-GATA1 (sc-265, Santa Cruz Biotechnology Inc.) or anti-Myc (LTK BioLaboratories) was preincubated with the nuclear extract on ice for 30 min before use in EMSA. Normal IgG was used as the control.

siRNA interference. The target sequences for siRNA interference of RREB1 mRNA (siRNA 1, 5′-GGGCAGACCUUUCAUACAGUU-3′; SEQ ID NO: 9; siRNA 2, 5′-GAAGAAAGCUGAUGAAGUCUU-3′; SEQ ID NO: 10) were identified using the manufacturer's design (DHARMACON®, ON-TARGETPLUS™). One strand of the control duplex RNA targeting the firefly luciferase mRNA is 5′-CUUACGCUGAGUACUUCGAUU-3′ (SEQ ID NO: 11). 2×10⁶ K562 cells were transfected with the duplex RNA oligos at the concentration of 100 nM. Using the NEON™ transfection system (INVITROGEN™), cells were microporated at 1,300V with a 30 ms width and one pulse, and then re-seeded in 2 ml antibiotics-free RPMI. 2 ml more of the medium were added to the cells 6 hrs later. After incubation for 48 hrs, half of the cells were harvested for assay of the gene expression. The remnants were re-microporated with siRNA oligos using the same conditions and incubated for another 48 hrs before assay.

Lentivirus-mediated knock-down experiments. Lentiviral plasmids (pLKO.1-shRNA) expressing short hairpin RNAs (snRNA 1, 5′-CCGGCCAGG AAACGAAAGAGGAGAACUCGAGUUCUCCUCUUUCGUUUCCUGGUUUUU-3′; SEQ ID NO: 12 and shRNA2, 5′-CCGGCGACGAUGACAAGAAAC CAAACUCGAGUUUGGUUUCUUGUCAUCGUCGUUUUU-3′; SEQ ID NO: 13) targeting the RREB1 mRNA were acquired from the TRC (The RNAi Consortium) lentiviral shRNA library. pLKO.1sh expressing a scramble shRNA and the shRNA-null puromycin-resistant vector (pLKO.1) were used to produce the control lentiviruses. The viruses were prepared by co-transfecting 293T cells the plasmid (pLKO.1-shRNA, pLKO.1sh, or pLKO.1), the packaging plasmid (pCMV-ΔR8.91), and the envelope plasmid (pMD.G). The culture medium containing lentiviruses were harvested at 64 hrs post-transfection for the estimation of the viral titer. For RNAi knockdown in K562 cells, spin-infection (MOI=2) was carried out at 2,750 g in 6-well plates for 30 minutes at 25° C., with a final concentration of 8 μg/ml of polybrene in the culture medium. After 24 hrs of lentiviral infection, the cells were selected with 2.5 μg/ml puromycin for another 4 days. The total RNAs were harvested from cells at 5 days and 10 days post-infection, respectively, for further quantitative RT-PCR analysis.

For RNAi knockdown experiments of primary human erythroid culture, the culture was initiated and prepared following the standard protocol except that STEMSPAN® SFEM medium (STEMCELL TECHNOLOGIES™) was used for culturing and maintenance of the cells. The cells were maintained in the differentiation medium at a density of 0.1˜1×10⁶ cells/ml. The lentivirus transductions were carried out on day 2 of the phase II culture of erythroid differentiation. Puromycin (2 μg/ml) selection was started at 24 hrs post-transduction for 9 days, and the total RNAs were then isolated using the RNAQUEOUS®-Micro Kit (AMBION™) for analysis by quantitative RT-PCR.

Quantitative RT-PCR analysis. RNAs from E9.5 mouse embryos with the yolk sac and the E14.5 mouse fetal liver were isolated with use of TRIZOL® reagent (INVITROGEN™). The RNAs of the RNAi knockdown cells were purified using the RNAspin Mini kit (GE HEALTHCARE™). cDNA synthesis was carried out using SuperScript II reverse transcriptase (INVITROGEN™). Quantitative RT-PCR was performed by using the SYBR® Green PCR Master Mix (APPLIED BIOSYSTEMS™) and ABI 7500 real-time System. All data were analyzed after normalization to the expression level of mouse Glycophorin A (GPA) or human β-actin gene. The sequences of the primers used for the quantitative RT-PCR are as follows: 5′hGH, 5′-TAGAGGAAG GCATCCAAACG-3′ (SEQ ID NO: 14); 3′hGH, 5′-GTCTGCTTGAAGATCTGCCC-3′ (SEQ ID NO: 15); 5′mGPA, 5′-GCCGAATGACAAAGAAAAGTTCA-3′ (SEQ ID NO: 16); 3′mGPA, 5′-TCA ATAGAACTCAAAGGCACACTGT-3′ (SEQ ID NO: 17); 5′hβ-Actin, 5′-CCTGAACCCCAAGGCCAACC-3′ (SEQ ID NO: 18); 3′hβ-Actin, 5′-CAGGGATAGCACAGCCTGGA-3′ (SEQ ID NO: 19); 5′RREB1, 5′-CGACTTAGGATTCACGGACTT C-3′ (SEQ ID NO: 20); 3′RREB1, 5′-CAGACAAAACGGTGTTG CTC-3′ (SEQ ID NO: 21); 5′hGATA1, 5′-TGGCCTACTACAGG GACGCT-3′ (SEQ ID NO: 22); 3′hGATA1, 5′-CATATGGTGAG CCCCCTGG-3′ (SEQ ID NO: 23); 5′hG3PDH, 5′-CAACTTTGGT ATCGTGGAAGGACTC-3′ (SEQ ID NO: 24); 3′hG3PDH, 5′-AGG GATGATGTTCTGGAGAGCC-3′ (SEQ ID NO: 25). Ruei-Lin Chen et al. (2010) “Developmental Silencing of Human ζ-Globin Gene Expression Is Mediated by the Transcriptional Repressor RREB1” THE JOURNAL OF BIOLOGICAL CHEMISTRY VOL. 285, NO. 14, pp. 10189-10197, which is herein incorporated by reference in its entirety.

### Results

Functional role of the ZF2 motif in erythroid cell cultures. The factor-binding motifs in the human ζ globin promoter region from −250 to −70, as determining previously by footprinting analysis in K562 nuclear extract, are displayed in FIG. 1A. Among these factor-binding motifs, ZF2 (−169 to −148) (FIG. 1A) consists of a GATA1 binding site followed by a sequence homologous to the consensus of the binding sites of the factor RREB1 (FIG. 1C). To examine the contributions of the putative GATA1 and RREB1 binding sites within the ZF2 motif to the ζ globin promoter activity, we introduced 3 different types of mutations (mT, mCC and 3 nt, FIG. 1C) into the ZF2 motif of the promoter. The activities of the wild type ζ promoter and the three mutants were then compared by hGH reporter (FIG. 1B) assay in transiently transfected K562 and MEL cells (FIGS. 1D-1E). As seen, the abolishment of the GATA1 binding site on ZF2 (mutant mT) caused significant reduction of the ζ promoter activity in K562 as well as in MEL cells (grey bars, FIGS. 1D-1E). On the other hand, mutation of the putative RREB1 binding site (mCC) resulted in approximately 2-fold higher promoter activity in either K562 or MEL cells (black bars, FIG. 1D-1E), but not in the non-erythroid 293T and HeLa cells in which the ζ globin promoter activities were very low (Table 1). Table 1 shows the expression levels (ng/ml) of hGH in transfected cells. Five micrograms of the two hGH expressing reporter plasmids were transfected into different types of cells. The amount (ng/mL) of hGH in the cell media at 48 hrs post-transfection was measured and normalized with β-galactosidase activity from the co-transfected plasmid pCMV-β gal. The elevated activity of the mCC mutant promoter in the erythroid cell lines was consistent with the previous study (Zhang, Q. et al. (1995) “Transcriptional Regulation of Human ζ2 and α Globin Promoters by Multiple Nuclear Factor-DNA Complexes: the Final Act” Molecular Biology of Hemoglobin Switch (Stamatoyannopoulos, G. ed., Intercept Limited, Andover, Hampshire, U.K.), further suggesting that factor(s) binding to the predicted RREB1 site of ZF2, possibly RREB1, was a repressor of the human ζ globin promoter.

TABLE 1

| | pBS-HS40-ζ-hGH | | | | pBS-HS40-α-hGH |
|---|---|---|---|---|---|
| Cell Types | WT | mT | 3nt | mCC | WT |
| K562 | 999 | 617 | 1,486 | 2,096 | 6,104 |
| MEL | 390 | 215 | 440 | 697 | 2,673 |
| 293T | 9.9 | 13.8 | 14.1 | 10.0 | — |
| HeLa | 3.9 | 10.6 | 8.5 | 3.4 | — |

Physiological role of ZF2 motif in transgenic mice. To further address the physiological role of ZF2 in the regulation of the ζ globin promoter, we analyzed transgenic mice carrying the same ζ-GH reporter fragments as used in FIG. 1B, with or without the mCC mutation (Table 2 and FIGS. 2A-2E). The positive lines were first identified by the presence of a 464 bp PCR fragment amplified from the human ζ promoter region (see Materials and Methods; data not shown). These lines were further analyzed by Southern blotting, as exemplified in FIG. 2A. The copy numbers of the transgene were determined and listed in Table 2. They varied from 1 to 60 for the wild type transgene and 1 to 40 for the mutant transgene (Table 2). As measured by the hGH assay of the plasma samples from the adult transgenic mice, the activities of the wild type human ζ globin promoter in most of the transgenic lines were very low, as also observed previously (Huang, B. L. et al. (1998) Proc Natl Acod Sci USA 95, 14669-14674). In contrast to the wild type, the mutant lines, except for line 251, showed significantly higher level of hGH per copy of the transgene. In particular, for the low copy lines, e.g., 221, 222 and 223, the levels of the plasma hGH per copy of the mutant transgene were 10-40 times higher than mice carrying the wild type promoter (Table 2). In Table 2, the lines with the same construct are grouped in the same column. The levels of the human growth hormone (hGH) in the plasma collected from the individual 3-month-old mice were determined by radioimmunoassay. The hGH level after normalization with the copy number are also listed.

TABLE 2

| | | hGH | Copy | | | | hGH | Copy | |
|---|---|---|---|---|---|---|---|---|---|
| Line | | (ng/ml) | no. | hGH/copy | Line | | (ng/ml) | no. | hGH/copy |
| Wt | 111 | 6.74 | 7 | 0.96 | mCC | 212 | 1.16 | 1 | 1.16 |
| | 112 | 0.28 | 2 | 0.14 | | 213 | 10.95 | 6 | 1.82 |
| | 121 | −0.32 | 10 | ND | | 221 | 8.06 | 2 | 4.03 |
| | 155 | 0.24 | 1 | 0.24 | | 222 | 25.71 | 2 | 12.85 |
| | 171 | 0.08 | 1 | 0.08 | | 223 | 2.31 | 2 | 1.16 |
| | 172 | 0.07 | 1 | 0.07 | | 231 | 6.03 | 11 | 0.55 |
| | 181 | 4.78 | 16 | 0.30 | | 244 | 1.78 | 5 | 0.36 |
| | 182 | 29.62 | 60 | 0.49 | | 251 | −0.20 | 4 | ND |
| | 191 | −0.31 | 5 | ND | | 281 | 169.60 | 12 | 14.13 |
| | | | | | | 282 | 104.35 | 40 | 2.61 |
| Non- | | −0.32 | | | | | | | |
| transgenic | | | | | | | | | |
| | | | | *0.07~1.0 ng/ml | | | | | 0.3~14 ng/ml |

*The asterisks indicates the range of the hGH levels per copy of transgene in the two groups of wt and mCC transgenic lines.

We have also analyzed the hGH expression in the mutant lines by RT-PCR analysis. First, to analyze the level in the adult mice, the mice were treated with phenylhydrazine to increase the erythropoiesis. RNAs were then isolated from the blood, spleen, liver, kidney and brain, and analyzed by semi-quantitative RT-PCR. As shown in FIG. 2C, for both the wild type and the mutant transgenic lines, the RT-PCR signals were detected mainly in the blood samples (lane B, FIG. 2C), although a minor signal could also be seen in the spleen samples (lane S, FIG. 2C). Finally, quantitative RT-PCR analysis showed that the mutant ζ globin promoter was also de-repressed in the yolk sac of E9.5 embryos and fetal liver of E14.5 embryos (FIG. 2D-2E). The data of FIG. 2 suggested that ZF2 also played a repressive role in the regulation of the human ζ globin promoter activity in vivo during erythroid development.

Factor-binding at the ZE2 motif. Following the above, we have used EMSA to examine the nature of the complex(es) formed on the ZF2 motif, in particular at the RREB1 sequence. For this, four oligos containing the wild type and the three different mutant ZF2 sequences listed in FIG. 1C were used as the EMSA probes (FIG. 3A). As shown in lanes 2-5 of FIG. 3B, four major complexes (a, b, G and ?) were present on the EMSA gels when the wild type (wt) ZF2 oligo was used. Of the four, band “?” was present only in K562 nuclear extract and it formed with all 4 oligos (FIG. 3B). This complex band was not studied further. Of the other three, complex “a” formed in all four nuclear extracts tested while complex “G” appeared only in the erythroid extracts (compare lanes 2 and 3 to lanes 4 and 5, FIG. 3B). Complex b was not present in the uninduced MEL extract (lane 3, FIG. 3B).

As described in FIG. 1, ZF2 consisted of two factor-binding sites, one for GATA1 and the other homologous to the RREB1 consensus binding sequence. To correlate the three EMSA complexes (a, b, and G) with these factors, oligos containing mutations at the GATA1 site (mT), the RREB1 sequence (mCC) and both (3 nt), respectively (FIG. 3A) were used in EMSA. As shown, when oligo ZF2 (mCC) was used, mainly band “a” disappeared (compare lanes 7 and 8 to lanes 2 and 3, FIG. 3B), suggesting that band “a” was a protein-DNA complex formed at the RREB1 consensus binding sequence. On the other hand, band “G” disappeared when oligo ZF2 (mT) was used (compare lanes 10 and 11 to lanes 2 and 3, FIG. 3B), suggesting that it was a GATA1/DNA complex. In the mean time, the amounts of both the complexes “a” and “b” increased in EMSA with the mT oligo (lanes 10 and 11, FIG. 3B). We interpreted the data of FIG. 3B as partly the result of the close proximity and possibly the overlapping nature of the RREB1 binding sequence and the GATA1 binding site (see sequence the oligo ZF2 (wt) in FIG. 3A). When GATA1 binding site was mutated, more bindings of factors, e.g. RREB1, to the RREB1 binding sequence and other yet-to-be determined factor-binding sites on the oligo became possible, thus forming more complexes “a” and “b”, respectively. In consistency with the EMSA data above, on the gel with use of the ZF2 (3 nt) oligo, in which both the GATA1 binding site and RREB1 sequence were mutated, complex “a” as well as complex “G” disappeared while complex “b” increased significantly (lanes 13 and 14, FIG. 3B).

The above suggested factor-binding scenario at the ZF2 motif in the K562 nuclear extracts was further confirmed by EMSA with ³²P-labeled probe(s), 50-200-fold molar excess of cold oligo competitors and supershift assays (FIG. 3C-H). For example, the formation of complex G with the ZF2 (wt) oligo as the probe was abolished when 100-fold excess of cold, GATA1-binding site-containing oligos, i.e. ZF2 (wt) (lane 2, FIG. 3C), ZF2 (mCC) (lane 5, FIG. 3C), and GATA1 (lanes 3 and 5, FIG. 3D), were pre-incubated with the nuclear extracts. That complex “G”, a GATA1/DNA complex, was also confirmed with the use of an anti-GATA1 antibody in a supershift assay (FIG. 3E). Similarly, the formation of the complex “a” was competed away with use of the ZF2 (wt) or ZF2 (mT) oligo as the competitor (lanes 2 and 3, FIG. 3C), but not by the ZF2 (3 nt) or ZF2 (mCC) oligo (lanes 4 and 5, FIG. 3C). More significantly, the formation of complex “a” on the wild type ZF2 oligo in the K562 nuclear extract could be effectively competed by an excess of either cold ZF2 oligo itself, or by cold RREB1-binding site-containing oligo (left panel, FIG. 3F), the latter of which was shown before to bind the RREB1 factor specifically. The reverse competitive EMSA experiment gave the similar result (right panel, FIG. 3F). Furthermore, the use of the oligos X1 or X2, both of which consisted of sequences unrelated to the RREB1-binding consensus, as the competitor could not abolish the formation of complex “a”, while the ZF2 and RREB1 oligos could (compare lanes 4 and 5 to lane 2 and lane 3, FIG. 3G).

That band “a” was a RREB1-DNA complex was further supported by a supershift assasy. Since no anti-RREB1 of supershift quality was available, we overexpressed Myc-tagged RREB1 by transient transfection of K562 cells with the plasmid pEF-Myc-RREB1 (lane 3 of the left panel, FIG. 3H). As seen in the right panel of FIG. 3H, pre-incubation of the Myc-RREB1-containing nuclear extract with anti-Myc resulted in the decrease of the intensity of band “a” (compare lanes 7 and 8 to lanes 5 and 6, FIG. 3H). The data of FIG. 3 together strongly suggested that RREB1 was the factor binding to the RREB1 binding sequence in the ZF2 motif.

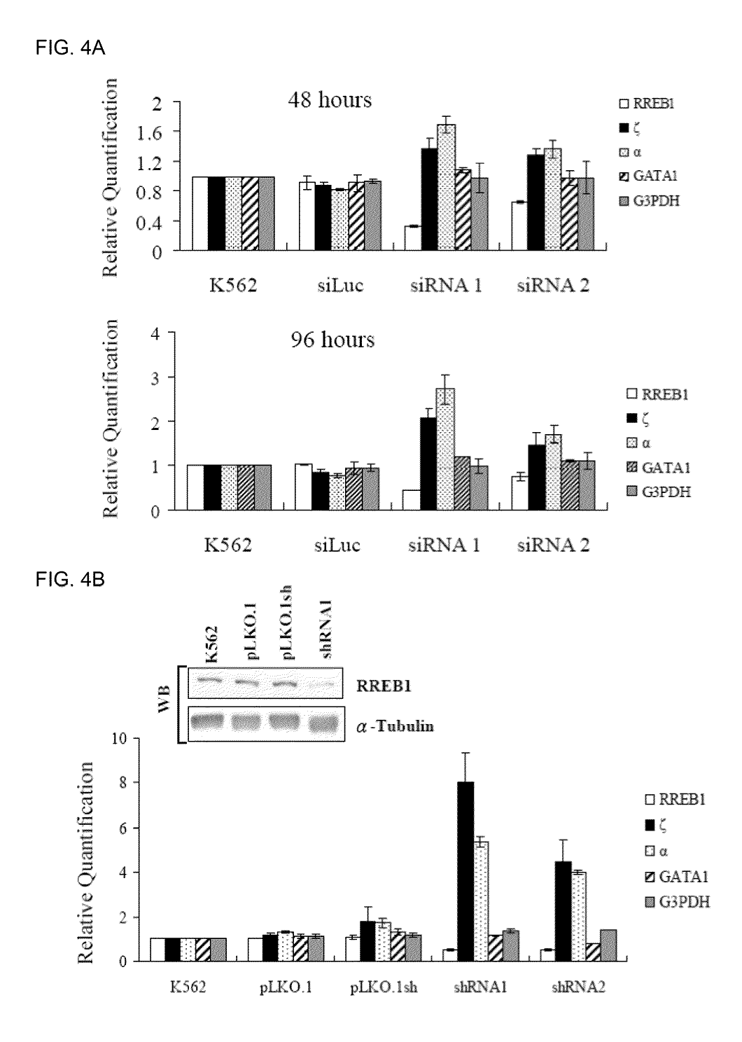

Elevated expression of the human ζ globin gene in RREB1-depleted cells. To further verify the negative regulatory effect of RREB1 on the ζ globin promoter activity, as suggested above by data from FIG. 1-3, we first knocked down the RREB1 expression in K562 cells by RNA interference (RNAi). For this, two different siRNA oligos were used, both of which were targeted at two specific sequences on the human RREB1 mRNA. After the RNAi treatment of K562 cells, the RNA was isolated and analyzed by quantitative RT-PCR. Of the two siRNA oligos, siRNA 1 reduced the level of RREB1 mRNA by 67% and siRNA 2 by 35% after 48 hrs of treatment. A small increase of the ζ globin mRNA (1.4 fold) was observed. To our surprise, reduction of RREB1 also caused an increase of the level of the α globin mRNA by approximately 1.7 fold, while those of the GATA1 mRNA and G3PDH mRNA were not altered (upper panel, FIG. 4A). At 96 hrs of the siRNA treatment, more increases of the two globin mRNAs were observed, with the ζ globin mRNA elevated by 2-fold and the α globin mRNA by 2.7-fold in siRNA 1 treated samples (lower panel, FIG. 4A).

We also analyzed the level of the ζ globin mRNA in K562 cells after more persistent reduction of the RREB1 expression with use of two different recombinant lentiviruses each expressing a short hairpin RNA (shRNA) targeting the RREB1 mRNA. The empty lentiviral vector was used as the control. As seen, the RREB1-shRNAs reduced the RREB1 mRNA levels to 40% after 5 days of infection (data not shown) and the knock-down of RREB1 mRNA could last for 10 days (FIG. 4B). Western blotting using an anti-RREB1 antibody from Rockland indicated that the RREB1 protein level was indeed lowered after knock-down of the RREB1 mRNA (panel in FIG. 4B). The ζ globin mRNA also remained at a high level after 10 days of infection, approximately 4-8 fold higher than the controls (FIG. 4B). Notably, the increases of the ζ and α globin gene expression, as shown in FIG. 4, were not by an indirect effect due to the induction of erythroid differentiation, since the mRNA levels of neither erythroid-related genes, e.g. GATA1 and NF-E2, nor non-erythroid genes, e.g. G3PDH, were significantly different between K562 cells with and without depletion of the RREB1 mRNA by RNAi knockdown (FIG. 4 and data not shown).

Finally, we have tested the effect of knocking-down of RREB1 expression by lentiviral-based shRNA in primary culture of adult human erythroid cells. While shRNA1 could not effectively knockdown the level of RREB1 mRNA in the primary culture (data not shown), expression of the lentiviral-based shRNA2 consistently reduced the RREB1 mRNA level by 50% (FIG. 4C). Interestingly, this reduction greatly increased the level of the ζ globin mRNA, by 70 fold, but not that of the α globin mRNA (FIG. 4C). As in RNAi-knockdown K562 cells, the levels of the GATA1 and G3PDH mRNAs were not altered either (FIG. 4C). These data of FIG. 4 strongly suggested the RREB1 played a repressive role in the human ζ globin gene expression in vivo.

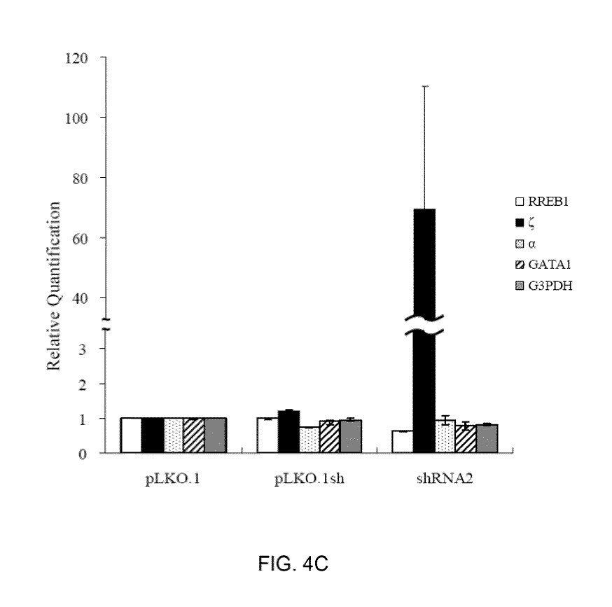

FIG. 5 shows Lentiviral-mediated knock-down of RREB1 mRNA in human erythroid K562 cells. Cells were infected with the indicated lentiviruses and then collected on the 10^{th} day after viral infection for RNA analysis by quantitative RT-PCR.

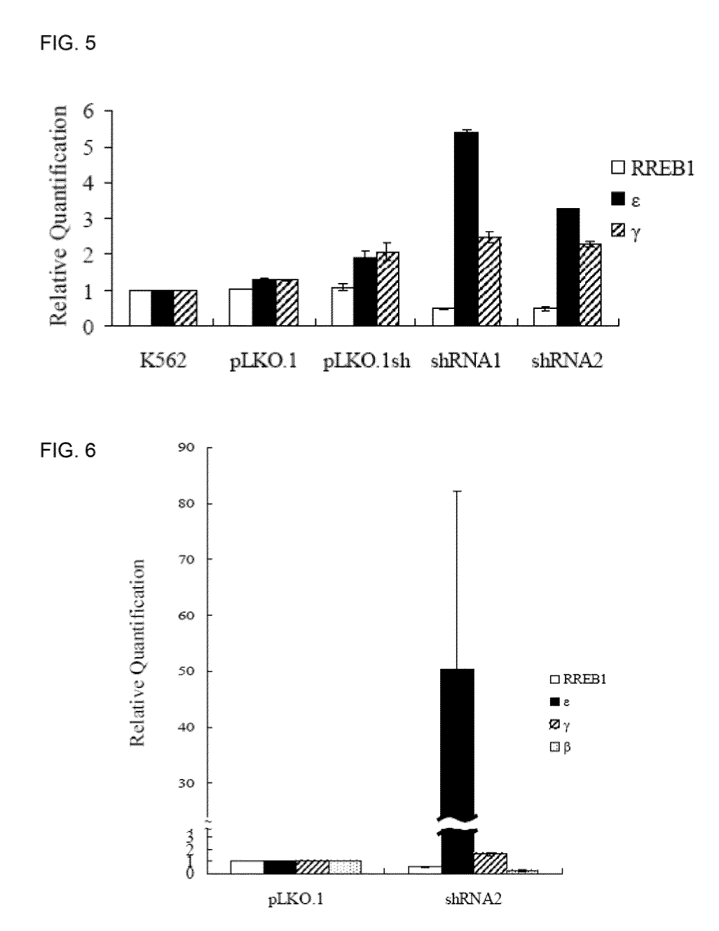

FIG. 6 shows Lentiviral-mediated knock-down of RREB1 mRNA in primary human erythroid cells. Primary cultures of human erythroid cells were infected with lentivirus carrying shRNA2 targeting the RREB1 mRNA. The total RNAs were isolated at 10^{th} day post-infection and subjected to quantitative RT-PCR analysis.

### Discussion

In this study, we have explored the possibility of re-turning on the human embryonic ζ globin gene at the adult stage by manipulating the formation of protein-DNA complex at a sequence motif, ZF2, in the ζ globin promoter region. We have also explored the identity of the factor(s) bound at ZF2 and repressing the ζ globin promoter activity. Our data suggest that binding of the factor RREB1 at ZF2 participates in the negative regulation of the ζ globin gene transcription during erythroid development.

Initially, the repressive role of ZF2 has been revealed from previous studies of mutagenized ζ globin promoter in K562 and MEL, both of which are well-established erythroid cell lines. The globin promoter activities from the different reporter plasmids were consistent with the cell-type and developmental-stage specificities of globin gene expression in the cell lines transfected. For example, the ζ globin promoter activity was lower in MEL than in K562, and it is extremely low in non-erythroid 293T and HeLa cells (Table 2). The role of ZF2 in K562 and MEL has been confirmed in the present study (FIG. 1). While mutation of the GATA1 binding site in ZF2 decreases the ζ promoter activity suggesting that GATA1 is an activator, the mCC mutation of the adjacent RREB1 sequence de-represses the ζ globin promoter in erythroid cell lines (FIG. 1D-1E) and in erythroid cells of the transgenic mice (FIG. 2). For the latter, the hGH levels in the blood samples of the transgenic mice carrying the wild type HS40-ζ-hGH construct are either very low or undetectable, except for line 182 (Table 1). The high copy number of the tandemly arranged transgenes in this line may have generated a novel transcription milieu that partially overcomes the silencing effect from the surrounding chromatin environment. On the other hand, the erythroid-specific activities of the mCC mutation-carrying human ζ globin promoter in different transgenic mouse lines at the adult stage are mostly 10-40-fold higher than the wild type ζ globin promoter (Table 2). The mutant promoter activity is also 7-10-fold higher than the wild type in E9.5 and E14.5 embryos (FIG. 2C). Note that the ζ→α hemoglobin switch already has occurred at E7.5, the earliest stage of erythroid development with manipulatable samples for experimentation.

The results from the DNA transfection studies in erythroid cell lines and transgenic mice analysis suggest that factor-binding at the RREB1 sequence in the ZF2 motif plays a key role in the silencing of the ζ globin promoter during erythroid development. RREB1 is an ubiquitously expressed, approximately 180 KD zinc finger protein that represses several other promoters, e.g. p16 and PSA, through binding to the RREB1 sites in these promoters. Furthermore, the repression by RREB1 is likely mediated through the RREB1-containing CtBP co-repressor complex. Although we have not been able to carry out chromatin-immunoprecipitation(ChIP) experiment due to the inaccessibility of appropriate anti-RREB1 antibody, several lines of evidence from our studies are highly suggestive that RREB1 is the factor, if not the only one, involved in the repression of the human ζ globin gene in vivo through direct binding at the ZF2 motif. First, the RREB1 sequence of ZF2 (FIG. 1C) is highly homologous to the binding consensus of RREB1, 5′-M-C-M-C-A-M-M-H-M-M-M-3′ (FIG. 1C), in which M is the nucleotide adenine or cytosine, and H is the nucleotide adenine, cytosine or thymine. Second, the mCC mutation at the CC di-nucleotides, which are conserved among all known and well-characterized RREB1 binding sites, of the RREB1 sequence on ZF2 abolishes its binding by the RREB1 factor, as suggested by the EMSA data (FIG. 3). At the mean time, the same C→G substitutions lead to the de-repression of the ζ globin promoter activity in erythroid cell lines and in the erythroid cells of transgenic mice (FIG. 1 and FIG. 2). Finally, RNAi knock-down of RREB1 level could elevate the mRNA level of the ζ globin gene in K562 cells as well as in primary culture of adult human erythroid cells (FIG. 4). With respect to the last result, it is interesting to note first that the α globin mRNA in the embryonic/fetal erythroid cell line K562 is also elevated upon knock-down of RREB1, and that there also exists a RREB1-binding site-like sequence (5′-GCCCCAGCCCAGCCCCGT-3′; SEQ ID NO: 26) in the α globin promoter at −674 to −661. More remarkably, RNAi knockdown of RREB1 expression in the adult human erythroid culture significantly elevates the level of the ζ globin mRNA but not the α globin mRNA. This suggests that RREB1 is involved in the silencing of the mammalian embryonic ζ globin promoter during the embryonic/fetal→adult erythroid development and, reciprocally, the repression of the α globin promoter at the early embryonic/fetal stages. Interestingly, RREB1 also behaved as a repressor of the ε globin gene transcription (FIGS. 5 and 6).

In summary, the data described in this study identifies RREB1 as a repressor involved in the developmental silencing of the human ζ globin gene, and likely that of other mammals as well. The repression of the embryonic ζ globin gene by RREB1 is in interesting analogy to the other two autonomously regulated human globin genes, i.e. the embryonic ε globin gene by YY1 and TR2-TR4, and the fetal γ globin gene by NF-E4 and BCL11A. The identification of RREB1 as a possible switch factor for the ζ globin gene expression provides a new research target for the treatment of certain forms of severe α-thalassemia.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A method of screening compounds capable of activating ζ and/or ε globin gene promoter activity in an erythroid cell, comprising:
(a) contacting in a medium a compound to be screened with Ras responsive element binding protein 1 (RREB1); wherein the medium comprises a polynucleotide comprising the nucleotide sequence of 5′-M-C-M-C-A-M-M-H-M-M-M-3′, wherein M is the nucleotide adenine or cytosine, and H is the nucleotide adenine, cytosine or thymine; the RREB1 bindable to the polynucleotide;
(b) determining binding of the compound to the RREB1; and
(c) determining change in binding of the RREB1 to the polynucleotide; wherein detection of binding of the compound to the RREB1 and change in binding of the RREB1 to the polynucleotide is indicative that the compound is capable of activating the ζ and/or ε globin gene promoter activity in the erythroid cell.

2. A method according to claim 1 for identifying a compound capable of increasing ζ and/or ε globin gene promoter activity in an erythroid cell, comprising detecting binding of the compound to be screened to the RREB1 and inhibition of binding of the RREB1 to the polynucleotide.

3. A method according to claim 2, wherein the compound is capable of increasing expression of two or more than two globin genes chosen from ζ globin gene, ε globin gene and α globin gene.

4. A method according to claim 1, wherein the compound is capable of increasing expression of two or more than two globin genes chosen from ζ globin gene, ε globin gene and α globin gene.

5. A method according to claim 4, wherein the compound is capable of increasing expression of ζ globin gene, ε globin gene and α globin gene.

6. A method according to claim 5 for screening antianemic agents.

7. A method according to claim 1 for screening antianemic agents.

8. A method according to claim 1 for screening agents for treating thalassemias and/or sickle cell anemia.

9. A method according to claim 1, wherein the polynucleotide is replaced by a DNA comprising the nucleotide sequence of SEQ ID NO: 26.

10. A method according to claim 9 for identifying a compound capable of increasing ζ and/or ε globin gene promoter activity, comprising detecting binding of the compound to be screened to the RREB1 and inhibition of binding of the RREB1 to the polynucleotide.

11. A method according to claim 9, wherein the compound is capable of increasing expression of two or more than two globin genes chosen from ζ globin gene, α globin gene and ε globin gene.

12. A method according to claim 11, wherein the compound is capable of increasing expression of ζ globin gene and α globin gene.

13. A method according to claim 1, wherein the polynucleotide is replaced by a DNA comprising the nucleotide sequence of SEQ ID NO: 28.

14. A method according to claim 13 for identifying a compound capable of increasing ζ and/or ε globin gene promoter activity in an erythroid cell, comprising detecting binding of the compound to be screened to the RREB1 and inhibition of binding of the RREB1 to the polynucleotide.

15. A method according to claim 13, wherein the compound is capable of increasing expression of two or more than two globin genes chosen from ζ globin gene, α globin gene and ε globin gene.

16. A method according to claim 15, wherein the compound is capable of increasing expression of ζ globin gene and α globin gene.

17. A method according to claim 1, wherein the compound is capable of increasing expression of ζ globin and ε globin genes.

18. A method of activating ζ and/or ε globin gene promoter activity in an erythroid cell comprising contacting the cell with a composition comprising a nucleic acid corresponding to the sequence of SEQ ID NO: 9, SEQ ID NO: 10, SEQ ID NO: 12, or SEQ ID NO: 13, thereby activating the ζ and/or ε globin gene promoter activity in the erythroid cell.

19. A method of treating a subject with thalassemias and/or sickle cell anemia comprising administering to the subject a vector expressing a nucleic acid corresponding to the sequence of SEQ ID NO: 12 or SEQ ID NO: 13, thereby treating the subject with thalassemias and/or sickle cell anemia.

20. The method of claim 19, wherein the vector is a lentiviral vector.
